(19) United States
(12) Patent Application Publication
(10) Pub. No.: US 2025/0295835 A1
(43) Pub. Date: Sep. 25, 2025

(54) BONE GRAFT SUBSTITUTES

(71) Applicant: ZOAN NUÁIL TEORANTA, Recess (IE)

(72) Inventors: Martin JOHNSON, Recess (IE); James MARTIN, Recess (IE); Stephen WANN, Recess (IE)

(21) Appl. No.: 18/728,744

(22) PCT Filed: Sep. 30, 2022
(86) PCT No.: PCT/EP2022/077409

§ 371 Date: Jul. 12, 2024

(30) Foreign Application Priority Data
Oct. 1, 2021 (GB) 2114126.2

(51) Int. Cl. A61L 27/36

(52) U.S. Cl. CPC A61L 27/3608; A61L 27/365; A61L 27/3683; A61L 2430/02; A61L 2430/40

(57) ABSTRACT

According to a first aspect, there is provided a bone graft substitute comprising coral particles. The coral material of the coral particles has a pore volume of below 15%. According to a second aspect, there is provided a bone graft substitute comprising coral particles, wherein the bone graft substitute has an overall pore volume comprising an inter-particle pore volume defined by voids between coral particles and an intra-particle pore volume defined by voids within coral particles, said overall pore volume being at least 40%, and said intra-particle pore volume being below 15%.

### BACKGROUND

The present disclosure relates to a bone graft substitute.

Bone grafting is a common transplant procedure. Autologous bone grafts can be considered the ideal graft material, as they provide osteoinductive and osteoconductive scaffolds with no immunogenicity and containing significant numbers of osteoprogenitor cells. However, autologous bone grafting has several drawbacks, including e.g. limited availability, variable graft quality, increased operative time and donor site morbidity. To overcome the increasing need for bone graft materials, research has focused on the development of new bone graft substitutes.

Various synthetic bone graft substitutes are known. Examples include ceramic products based on calcium phosphate, such as hydroxyapatite and tricalcium phosphate. Bone graft biomaterials derived from mineralizing marine organisms have also been investigated. Several marine species produce mineralized structures within their anatomy that resembles the human bone. Examples of such species include sponges (Porifera), red algae (Rhodophyta), corals (Cnidarians) and a range of other organisms like snails (Mollusca), starfish (Echinodermata). Among such marine derived biomaterials, corals are one of the most studied in the field of bone tissue engineering.

Studies have revealed that some corals have significant structural similarities to cancellous bone. Many types of coralline material have been characterized by a network of interconnected channels and pores. When implanted in-vivo, some coral implants have been found to be biocompatible, allowing vascular ingrowth and inhabitation of cell lineages found in bone. However, among the different coral species (there are approximately 7000 species of coral), significant structural differences exist. This can have direct implications to their bone-forming capacity.

To date, the main species investigated for bone graft applications are Acropora sp., Goniopora sp. and Porites sp. (Injury, Int. J. Care Injured 47 (2016). Other coral genera have been previously investigated but with limited use. Among them, corals of the genus, Dichocoenia sp., were found to trigger a foreign-body reaction when implanted in rabbits. These corals were also found to have slow resorption rates. Lobophyllia sp and Pocillopora sp have a skeletal structure similar to the diaphysis of compact bone with a dense and compact outer wall (theca) surrounded by a thin inner septa. It has been previously proposed that the larger the pore volume of the coral material, the greater the coral resorption as well as the new bone apposition (Injury, Int. J. Care Injured 47 (2016)). With their compact structure, such corals have not been extensively studied for bone graft applications.

### BRIEF DESCRIPTION OF FIGURES

Features of the present disclosure are described, by way of examples, with respect to the accompanying figures, in which:

FIG. 1 shows SEM images showing the surface topology of Pocillopora coral (see Example 2);

FIG. 2 shows SEM images of mesenchymal stem cells grown on Pocillopora coral (Example 5);

FIG. 3 shows representative photomicrographs of implant/void sites after intra-osseous implantation of a bone graft substitute according to an example of the present invention in rabbits after 26 weeks (Example 7); and

FIG. 4 compares the bone+implant volume % values for various implants following intra-osseous implantation in rabbits after 4, 13 and 16 weeks (Example 7).

### DETAILED DESCRIPTION

According to a first aspect, there is provided a bone graft substitute comprising coral particles. The coral material of the coral particles has a pore volume of below 15%.

According to a second aspect, there is provided a bone graft substitute comprising coral particles, wherein the bone graft substitute has an overall pore volume comprising an inter-particle pore volume defined by voids between coral particles and an intra-particle pore volume defined by voids within coral particles, said overall pore volume being at least 40%, and said intra-particle pore volume being below 15%.

According to a third aspect, there is provided a bone graft substitute comprising coral particles, wherein the bone graft substitute has an overall pore volume comprising an inter-particle pore volume defined by voids between coral particles and an intra-particle pore volume defined by voids within coral particles, wherein the overall pore volume is at least 3 times greater than the intra-particle pore volume.

According to a fourth aspect, there is provided a method of manufacturing a bone graft substitute as described herein, wherein said method comprises:
(i) growing coral in a growth medium;
(ii) removing at least a portion of the coral from the growth medium;
(iii) devitalising coral removed from the growth medium; and
(iv) sizing the devitalised coral to form particles for use as the bone graft substitute.

According to a preferred aspect, there is provided a bone graft substitute comprising coral particles. The coral particles comprise coral material of the genus Pocillopora. The coral material of the coral particles has a pore volume of below 15%.

The bone graft substitute of the present invention has been found to be an effective bone graft substitute, providing an effective scaffold for bone repair. Coral material, preferably of the genus Pocillopora, has improved biocompatibility compared to, for example, commercially available bone graft substitutes based on synthetic materials. The surface topology of the coral material may also be conducive to cell adhesion and proliferation.

The coral particle has a pore volume of below 15%. As a consequence, the pore volume of the individual coral particles may be relatively low. Although the porosity may be low, the surface topology of the coral (preferably, Pocillopora) can nevertheless provide an effective scaffold for bone growth because of the biocompatibility and surface topology of the coral material.

Furthermore, the low pore volume of the coral particles (intra-particle pore volume) can provide increased mechanical strength, and a reduced rate of resorption e.g. compared to previous coral-based bone grafts. As the bone graft substitute integrates into new bone growth, the reduced rate of resorption and/or improved mechanical strength can facilitate the formation of a matrix having improved load-bearing characteristics. This may reduce the risk of e.g. fracture at the grafting site before bone growth is complete. This may also reduce the risk of e.g. fracture that may arise from poor remodeling of the bone, for example, when resorption occurs too rapidly. The biocompatibility of the coral, preferably Pocillopora coral, may also reduce the risk of adverse foreign-body reactions that may otherwise arise from the reduced rates of resorption. Moreover, because of the biocompatibility of the coral (e.g. Pocillopora coral) the bone graft substitute may provide improved osseointegration, facilitating improved direct contact between living bone and the surface of the particles.

In some examples, the bone graft substitute may be in granule form (i.e. particulate form). The bone graft substitute may have an overall pore volume that includes an inter-particle pore volume defined by voids between coral particles and an intra-particle pore volume defined by voids (if any) in the coral material of the particles themselves. The intra-particle pore volume refers to the pore volume of the coral material of the coral particles. The inter-particle voids are open or interconnected and can facilitate osteoconduction, allowing bone to grow through the scaffold. When implanted, bone-forming cells in the grafting area can move across the scaffold and slowly replace the scaffold with new bone over time. While the pore volume of the coral material of the coral particles (intra-particle pore volume) may be less than 15%, the overall pore volume of the bone graft substitute may be higher, owing, for example, to a higher pore volume arising from e.g. voids between the coral particles (inter-particle pore volume).

As mentioned above, the pore volume of the coral material of the coral particles (intra-particle pore volume) is less than 15%. The pore may be less than 10%, preferably, less than 8%, more preferably, less than 6%. In some examples, the pore volume of the coral material of the coral particles may be less than 5%, for instance, less than 3%. In some examples, the pore volume of the coral material of the coral particles may be 0% or greater, preferably, at least 0.2%, more preferably at least 0.5%, and even more preferably at least 0.7%. In some examples, the pore volume of the coral material of the coral particles may be 0 to 15%, preferably 0.2 to 10%, more preferably 0.5 to 8%, even more preferably, 0.7 to 6%. In some examples, the pore volume of the coral material of the coral particles may be 1 to 3%.

Intra-particle pore volume may be measured by mercury porosimetry. Mercury porosimetry is used to measure the open or interconnected pore volume of a material by applying controlled pressure to a sample immersed in mercury. External pressure is required for mercury to penetrate into the pores of a material due to high contact angle of mercury. The amount of pressure required to intrude into the pores is inversely proportional to the size of the pores. The larger the pore the smaller the pressure needed to penetrate into the pore.

Mercury porosimetry is based on the capillary law governing liquid penetration into small pores. This law, in the case of a non-wetting liquid like mercury, is expressed by the Washburn equation:

$$D = ( 1 / P ) 4 γ cos ω$$

where D is pore diameter, P is the applied pressure, γ is the surface tension of mercury and ω is the contact angle between the mercury and the sample. The volume of mercury V penetrating the pores is measured directly as a function of applied pressure.

As pressure increases during an analysis, pore size is calculated for each pressure point, and the corresponding volume of mercury required to fill these pores is measured. The measurements are taken over a range of pressures to give the pore volume for the sample material.

Mercury porosimetry may be useful for determining the pore volume within the coral material of the coral particles (i.e. the intra-particle porosity). An example of a suitable mercury porosimetry method is ASTM UOP 578-11.

Mercury porosimetry may also be used to determine the total pore area (intra-particle pore area), and average pore diameter (intra-particle average pore diameter) of the coral material of the coral particles. This excludes the pore area and average pore diameter associated with the inter-particle pores arising from voids between particles.

The pore area of the coral material of the coral particles (intra-particle pore area) may be up to 0.5 m²/g, preferably 0.01 to 0.4 m²/g, more preferably 0.05 to 0.35 m²/g, even more preferably 0.1 to 0.3 m²/g, for instance, 0.15 to 0.25 m²/g.

The average (mean) pore diameter of the coral material of the coral particles (intra-particle pore diameter) may be less than 1 μm, preferably less than 0.5 μm. The average (mean) pore diameter may be 0.01 to 0.4 μm, preferably 0.05 to 0.3 μm, for example, 0.1 to 0.2 μm.

The pore volume, pore area and/or pore size of the coral material of the coral particles may be varied to control the mechanical strength and/or resorption rate of the bone graft substitute. In some instances, for example, the pore volume and/or pore area of the coral material of the coral particles may be reduced to improve mechanical strength and/or resorption rate of the scaffold. The pore volume and/or pore area of the coral material may be controlled by varying the growing conditions of the coral. For example, as will be explained in further detail below, the coral may be grown (e.g. in captivity) in a growth medium having a carbonate hardness, dKH, of 8 or more. In some instances, by increasing carbonate hardness, dKH to 8 or above, the pore volume and/or pore area of the coral may be varied e.g. reduced. In some instances, the carbonate hardness can be controlled at dKH of 8 or above. Preferably, the growth medium has a carbonate hardness, dKH, of 10 or 10.5 to 14, for example, 12 to 14. More preferably, the growth medium has a carbonate hardness, dKH, of 13 or greater, for example, 13 to 14 or about 13.5. The carbonate hardness may be controlled at a dKH of 10 to 14, preferably 12 to 14.

In some examples, the carbonate hardness may be controlled to within ±3 dKH units. In some examples, the carbonate hardness may be controlled to within ±2 dKH units, preferably to within ±1.5 dKH units or ±1 dKH units. More preferably, the carbonate hardness may be controlled within ±0.5 dKH units.

Intra-particle pore volume and/or intra-particle pore area may also be controlled by harvesting specific portions of coral. Coral may be harvested from different parts of the coral depending, for example, on the age or maturity of the coral at that location. Coral skeletons may comprise a core or trunk, with branches extending from the core or trunk. Coral may be harvested from the core or trunk, or from branches. Where coral is harvested from branches, the distance of the location of harvest from the branch tip and/or branch base may be varied depending on desired characteristics of the harvested coral. In some instances, the coral may be harvested from branches of coral e.g. branches of coral of a threshold size range and/or of a specified level of maturity. In some instances, the coral may be harvested from the coral core. The site from which coral is removed may, in some instances, have a bearing on the pore volume of the coral material. As a result, this may also have a bearing on the intra-particle pore volume of e.g. any coral particles of the bone graft substitute produced. The harvested coral may have a desirable mechanical strength and/or with a structure similar to that of compact or cortical bone. In the case of granules, the manner in which the harvested coral is milled or processed may also have an influence on the intra-particle pore volume of the coral particles of the bone graft substitute produced.

The bone graft substitute may have an overall pore volume comprising an inter-particle pore volume defined by voids between coral particles and an intra-particle pore volume defined by voids (if any) in the coral material of the particles themselves. The inter-particle voids are open or interconnected and can facilitate osteoconduction, allowing bone to grow through the scaffold. When implanted, bone-forming cells in the grafting area can move across the scaffold and slowly replace the scaffold with new bone over time. In an instance, the pore volume of the coral material of the coral particles (intra-particle pore volume) is less than 15% and the overall pore volume of the bone graft substitute is at least 40%.

The bone graft substitute of the present invention may have an overall pore volume of greater than 40%. For example, where the bone graft substitute comprises coral particles (e.g. coral granules), the coral particles may have an overall pore volume of greater than 40%. The overall pore volume may include the pore volume associated with voids between particles (inter-particle pore volume) and the pore volume associated with voids (if any) in the coral material of the coral particles (intra-particle pore volume). In some examples, for instance, where the pore volume of the coral material of the coral particles is low, the overall pore volume may approximate the inter-particle pore volume.

Preferably, the overall pore volume may be at least 45%, more preferably, at least 50%, for example, at least 55%. The overall pore volume may be at most 80%, preferably at most 75%, more preferably at most 70%, for example, at most 65%. In some examples, the overall pore volume may be 40 to 80%, preferably 45 to 75%, more preferably 50 to 70%, for instance, 55 to 65%.

The overall pore volume may be determined by determining the tapped density of the sample. For example, where the bone graft substitute is a particulate composition (e.g. granules), the overall pore volume may be measured by inserting a volume of the sample into a test container having a known volume. The test container may be tapped several times (e.g. 1000 times) using, for example, a pneumatic device to ensure that the coral scaffold settles into the test container. Excess sample can be removed from the top of the container to ensure that the container is filled. By dividing the mass of the filled container by the known volume of the container, the tapped density, ρ tapped, may be obtained. The overall pore volume, P, may be calculated from the tapped density as follows:

$$P = [ 1 - ( ρ tapped / ρ theoretical ) ] \times 100$$

where ρ theoretical is the theoretical density of the coral scaffold material (e.g. aragonite, ρ theoretical=2.93 g/cm³).

Where the bone graft substitute is in particulate form (e.g. granules), the overall pore volume or overall porosity may be determined from the tapped and theoretical densities may be indicative of the porosity within (intra-particle) and between the particles (inter-particle) of particles of the bone graft substitute. Where the intra-particle porosity is low relative to overall porosity (less than 10% for example) the overall pore volume represents to first order the inter-particle pore volume.

An example of a suitable method for determining overall pore volume is ISO 23145-1:2007.

In some instances, the closed pore volume of the coral may be low or negligible. (This can be confirmed by e.g. visual inspection of images (e.g. micrographs) of the coral material). The intra-particle pore volume of the coral material is, therefore, the intra-particle open pore volume. In such examples, the inter-particle pore volume can be calculated or approximated by the following equation, as follows:

$${Φ}_{INTER} = ( {Φ}_{T O T -} {φ}_{INTRA\_OPEN} ) / ( 1 - {φ}_{INTRA\_OPEN} )$$

Where:
Φ_{TOT} is the total porosity as calculated by the tapped density method above and is equivalent to the overall pore volume or porosity, P as described above, and
ϕ_{INTRA_OPEN} is the intra-particle porosity expressed as a fraction of particle volume, measured using mercury porosimetry as described in herein.

In some examples, for instance, where the bone graft substitute is particulate (e.g. in the form of granules), the particles of the bone graft substitute may have a tapped density of at least 600 mg/cm³, preferably at least 700 mg/cm³, more preferably at least 800 mg/cm³, yet more preferably at least 900 mg/cm³. The particles of the bone graft substitute may have a tapped density of at most 1500 mg/cm³, preferably at most 1300 g/cm³, more preferably at most 1200 g/cm³, yet more preferably at most 1100 g/cm³. In some examples, the particles of the bone graft substitute may be 600 to 1500 g/cm³, preferably 700 to 1300 g/cm³, more preferably 800 to 1200 g/cm³, yet more preferably 900 to 1100 g/cm³. In one example, the particles of the bone graft substitute may be 1000 to 1100 g/cmcm³.

In some examples, for instance, where the bone graft substitute is particulate (e.g. in the form of granules), the bone graft substitute may have an inter-particle pore volume defined by voids between particles of greater than 30%. Preferably, the inter-particle pore volume may be at least 40%, more preferably, at least 45%, for example, at least 50%. The inter-particle pore volume may be at most 75%, preferably at most 70%, more preferably at most 65%. In some examples, the overall pore volume may be 30 to 75%, preferably 40 to 70%, more preferably 45 to 65%, for instance, 50 to 65%. The inter-particle pores may be suitable to facilitate cell adhesion, aggregation, in-growth and proliferation, while at the same time providing sufficient space for vascularization for adequate nutrient and oxygen supply. The inter-particle pore volume may be selected so that the bone graft substitute may be suitable for use as a bone engineering scaffold.

The inter-particle pore volume (and hence the overall pore volume) may depend on the size of the particles of coral. The inter-particle pore volume (and hence the overall pore volume) may also depend on the particle size distribution of the particles. The particle size and particle size distribution may be varied by varying the manner and/or extent to which the particles are sized (e.g. by grinding).

In some examples, the overall pore volume is at least 3 times greater than the intra-particle pore volume. The overall pore volume may be at least 5 times, for example, 8 to 50 times or 10 to 40 times greater than the intra-particle pore volume.

The bone graft substitute may comprise coral particles that have a mean particle size of 0.005 to 8 mm, for example, 0.01 to 6 mm. The mean particle size may be selected depending on how the coral particles are intended to be used. For example, where the bone graft substitute takes the form of a 3D printing composition, the particles may be relatively small in size, for example, below 100 μm or below 80 μm, for instance, 5 to 30 μm. Where the bone graft substitute takes the form of a putty, the particle size may be less than 100 μm, for example, 30 to 80 μm. In the case of granules, the particle size may be 0.1 to 5 mm, preferably, 0.5 to 4 mm, more preferably 0.7 to 3 mm, yet more preferably 1 to 2 mm.

In some examples e.g. where the bone graft substitute takes the form of granules, at least 75 wt. % of the coral particles have a particle size of 1.0 to 2.0 mm. For example, at least 80 wt. % or at least 85 wt. % of the coral particles have a particle size of 1.0 to 2.0 mm. In some examples, 75 to 100 wt. %, preferably 80 to 97 wt. %, more preferably 85 to 95 wt. % of the coral particles have a particle size of 1.0 to 2.0 mm. At least 45 wt. % of the coral particles may have a particle size of 1.4 to 2.0 mm. For example, at least 50 wt. % of the coral particles have a particle size of 1.4 to 2.0 mm. In some examples, at least 55 wt. % of the coral particles have a particle size of 1.4 to 2.0 mm. 55 to 65 wt. % of the particles may have a particle size of 1.4 to 2.0 mm. At least 20 wt. % of the coral particles may have a particle size of 1.0 to 1.4 mm. For example, at least 25 wt. % of the coral particles may have a particle size of 1.0 to 1.4 mm. In some examples, at least 30 wt. % of the coral particles may have a particle size of 1.0 to 1.4 mm. 30 to 45 wt. % of the particles may have a particle size of 1.0 to 1.4 mm. Fewer than 10 wt. % of the particles have a particle size of greater than 2.0 or less than 1.0 mm. In some examples, fewer than 8 wt. % of the particles have a particle size of greater than 2.0 or less than 1.0 mm. Fewer than 5 wt. % of the particles have a particle size of greater than 2.0, and fewer than 5 wt. % have a particle size of less than 1.0 mm.

In some examples e.g. where the bone graft substitute takes the form of granules, the particle size distribution of the coral particles may be as follows:

| Size range (mm) | Weight fraction in % |
|---|---|
| d < 1.0 | 3.6 ± 0.5 |
| 1.0 ≤ d < 1.4 | 32.3 ± 3.4 |
| 1.4 ≤ d < 2.0 | 60.5 ± 3.1 |
| D ≥ 2.0 | 3.6 ± 0.1 |

Particle size distribution may be determined by sieving. A suitable method is described under DIN 66165-2:2016-08 with sieves manufactured to ISO 3310-1:2016 and ASTM E11 can be used.

In the case of granules, the inter-particle pore volume may be varied by varying the particle size and/or particle size distribution of the granule sample. This may be varied to facilitate cell-ingrowth into the wound site. In the case of granules, the inter-particle pore volume may comprise pores having an average pore size of 100 to 800 μm. In some examples, the pores may be 100 to 500 microns in size, preferably 100 to 325 microns in size. The pore size may be suitable to facilitate cell in-growth, while at the same time providing sufficient space for vascularization for adequate nutrient and oxygen supply. The pore size may be selected so that the bone graft substitute may be suitable for use as a bone engineering scaffold.

In some examples, the coral particles may have a surface roughness that causes the particles to aggregate or adhere to one another. In some examples, the coral particles have a surface roughness that reduces the flowability of the particles. This tendency to adhere or aggregate may, in some examples, contribute to the mechanical strength of the bone graft substitute. As discussed above, the surface topography of the coral particles may also facilitate, for example, cell adhesion and support cell proliferation and growth across the scaffold.

The bone graft substitute may further comprise a therapeutic agent. The bone graft substitute may be used to deliver the therapeutic agent at the site of implantation. Examples of suitable therapeutic agents include antimicrobials, anti-inflammatory agents and/or cancer drugs.

The bone graft substitute may further comprise an antimicrobial and/or anti-inflammatory agent. The agent(s) may be present, for example, as a coating on the particles. For example, the agent(s) may coat the surface of the pores of the bone graft substitute. Any suitable anti-inflammatory agent may be employed. The anti-inflammatory agent may reduce inflammation at the site of implantation. Any suitable antimicrobial agent may be employed. For example, the antimicrobial agent may be an antibiotic. The antimicrobial agent may reduce the risk of infection at the site of implantation.

The bone graft substitute may be provided in the form of granules. Alternatively, the particles are provided in the form of a putty. The putty may further comprise a binder, for example a cellulose binder. The putty may be applied using, for example, a syringe for easier application to repair osteoporotic fractures and for spinal applications.

The bone graft substitute may be provided as a 3D printing composition mixed with other components such as binders or crosslinking agents. For example, the 3D printing composition may be in the form of an ink for extrusion through a print nozzle. The 3D composition could alternatively be in the form of a slurry or paste that may be printed using a lithographic 3D printer. Alternatively, the 3D printing composition may take the form of 3D build material that may be bound into a desired shape or form by selective application of a binder.

In some examples, the bone graft substitute may be used as a coating, for example, on a bone implant (e.g. a metal bone implant). In some examples, the bone graft substitute may be used to fill voids in and around e.g. a bone implant (e.g. a metal bone implant).

In some examples, the bone graft substitute may be used in combination with a different tissue scaffold. For example, the bone graft substitute may be used in combination with a scaffold for supporting the growth of e.g. collagen, cartilage, skin or muscle tissue.

In some examples, the bone graft substitute may be treated with protein, growth factor or cells (e.g. stem cells) to facilitate bone growth. For example, the bone graft substitute may comprise osteoinductive factors including human bone morphogenic protein 2 (h-BMP2). Other examples of factors include transforming growth factor β (TGF-β), platelet-derived growth factor (PDGF) and bioactive polypeptides. The protein, growth factor or cells may be present as a matrix within the porous structure of the bone graft substitute. The bone graft substitute may be used in combination with allograft tissue, and/or endogenous bone forming cells including mesenchymal stem cells, osteoprogenitor cells and osteoblasts as well as osteoinductive and angiogenic growth factors.

The bone graft substitute may be suitable for use as an osteoconductive matrix. In some examples, the coral scaffold may be used as anosteoconductive matrix for cancellous bone tissue. In some examples, the coral scaffold may be used as an osteoconductive matrix for cortical bone tissue. The bone graft substitute may be suitable for impaction grafting. The bone graft substitute may be suitable for bone re-building, for example, following tumour removal and/or revision of artificial joints. The bone graft substitute may also be used for filling voids or other bone defects.

The bone graft substitute may be used as a scaffold for bone growth in any bones in humans or other mammals including the spine, sternum, ribs, femur, tibia, fibula, talus, patella, ulna, radius, humerus, pelvis, scapula, skull, jawbone and teeth.

The coral particles (e.g. coral particles of the coral genus Pocillopora) may be used as an extender for an autograft or allograft bone. For example, the bone graft substitute may further comprise allograft and/or allograft bone fragments in admixture with the particles of the coral.

The coral particles may be combined with bone marrow e.g. prior or during the implantation process. In some examples, the bone graft substitute may further comprise bone marrow in combination with the coral particles.

The coral material used to form the bone graft substitute may have a compressive strength of at least 8 MPa. The coral material may have a compressive strength of at least 9 MPa, preferably at least 10 MPa. The coral material may have a compressive strength of at most 200 MPa. In some examples, the coral material may have a compressive strength of at least 12 MPa, for example, at least 15 MPa or at least 20 MPa or preferably at least 40 MPa, for example, at least 45 MPa, at least 50 MPa, at least 55 MPa or at least 60 MPa. Compressive strength may be measured by any suitable method. For example, a test piece of the coral material used to form the coral particles of the bone graft substitute may be compressed between the platens of a compression-testing machine by a gradually applied load. The load under which the test piece cracks is used to determine the compressive strength of the coral material. The test piece may be shaped in any suitable form. For example, in one embodiment, the test piece may be shaped as a cylinder, for example, with a L/D (length to diameter ratio) of e.g. 1.5. Measurements may be formed using a 5 kN load cell at a loading rate of 1 mm/min. A suitable method is described in ISO 9917.

The coral material may have a compressive strength that is higher than cancellous bone. The coral material may have a compressive strength that is comparable to or greater than that of compact or cortical bone.

### Pocillopora

As described above, the bone graft substitute of the present invention may comprise particles of the coral genus Pocillopora. Preferably, the Pocillopora is of the species:
Pocillopora acuta
Pocillopora aliciae
Pocillopora ankeli
Pocillopora bairdi
Pocillopora brevicornis
Pocillopora capitata
Pocillopora damicornis
Pocillopora danae
Pocillopora effusus
Pocillopora elegans
Pocillopora eydouxi
Pocillopora fungiformis
Pocillopora indiania
Pocillopora kelleheri
Pocillopora ligulata
Pocillopora meandrina
Pocillopora molokensis
Pocillopora verrucosa
Pocillopora woodjonesi
Pocillopora zelli

In some examples, the Pocillopora may be Pocillopora damicornis.

Preferably, the coral (e.g. Pocillopora) is grown in captivity. Coral (e.g. Pocillopora) can be grown in captivity under controlled conditions. The growing conditions can be controlled to vary the characteristics of the coral, including the intra-particle pore volume. Growth conditions, such as temperature, light intensity, pressure, pH and carbonate hardness of the growth medium can be controlled independently or in combination with one another to vary characteristics of the coral. For example, one or more of such growth conditions may be controlled to obtain desired intra-particle pore volumes.

Corals grown in captivity may also be more consistent in structure, with e.g. lower levels of contaminants. Corals grown in captivity may also have advantages in terms of sustainability, as the harvesting of wild coral can have a negative impact on sustainability and ocean biodiversity.

In a further aspect, there is provided a method of manufacturing the bone graft substitute as described herein. The method comprises growing coral in a growth medium (e.g. having a carbonate hardness, dKH, of 8 or more); removing at least a portion of the coral from the growth medium; devitalising coral removed from the growth medium and sizing the devitalised coral to form particles for use as the bone graft substitute.

In the method of the present invention, coral is grown in a growth medium having a carbonate hardness, dKH, of 8 or more. By controlling the carbonate hardness at such elevated levels, it has been found that the porosity or pore volume of the coral can be varied. As well as porosity, the pore size distribution and/or the pore structure may also be altered. This may allow the mechanical properties of the coral to controlled.

For example, the pore volume in the coral can be varied to improve the properties of the bone graft substitute. In some instances, decreases in pore volume may improve the mechanical strength of the bone graft substitute. This, in turn, may allow the material to be used in a broader range of applications, for example, where improved load bearing may be required. In some examples, a change in pore volume of the coral may result in changes in the way in the bone graft substitute is resorbed by the body.

### Growth Medium

The coral may be grown in any suitable growth medium. The growth medium may be an aqueous medium. The aqueous medium may be a saline solution. Examples of suitable growth media include freshwater, seawater and mixtures thereof. In some examples, a mixture of seawater and freshwater may be employed. In other examples, the growth medium may comprise a saline solution formed by dissolving sodium chloride and, optionally, other salts in water. The water may be filtered prior to use. In some examples, the growth medium may be circulated or agitated. This circulation or agitation may simulate the circulation of water that coral may be exposed to in its natural environment. The rate of circulation or agitation may be controlled to vary characteristics of the coral (for example, to obtain desired intra-particle pore volumes). Suitable rates of circulation can be a linear flow provided within a range of 0 to 30 cm/s, preferably 2-15 cm/s.

In some examples, the salinity of the growth medium may be controlled. In some examples, the salinity may be controlled by varying the relative amounts of saltwater (e.g. seawater) and freshwater in the growth medium. Salinity may be controlled such that the specific gravity of the growth medium is from 1.0 to 1.1, preferably from 1.022 to 1.032 at 25 degrees C. In some examples, the salinity may be controlled from 1.024 to 1.026 at 25 degrees C., for instance, at about 1.025. In some examples, the method comprises controlling salinity within ±10%, for example, ±8%, ±6%, ±5%, ±4%, ±3%, ±2%, ±1%, ±0.5% of a target specific gravity value. The target specific gravity may be 1, for example, 1.02 or 1.03.

The growth medium may have a carbonate hardness, dKH, of 5 or more, for example, 8 or more. Carbonate hardness is a measure of the water hardness caused by the presence of carbonate (CO₃²⁻) and bicarbonate (HCO₃⁻) ions. The carbonate hardness is indicative of the extent to which the growth medium is buffered with respect to changes in pH. By controlling the carbonate hardness, the rate and nature of coral growth can be varied. It has surprisingly been found that, at levels of carbonate hardness of 8 or more, the rate of coral growth can be enhanced. It has also been found that this increased rate in coral growth affects the mechanical properties and structure of the resulting coral. For example, the resulting coral has a different porosity. In some examples, the difference in porosity may result in a different pore structure and/or a different pore size distribution. In some instances, the coral may have a lower porosity and improved mechanical strength (e.g. compressive strength).

In some examples, the dKH of the growth medium is controlled at a dKH value of 8 or more within ±2 dKH units. In some examples, the dKH of the growth medium is controlled at a dKH value of 8 or more within ±1.5 dKH units, preferably within ±1 dKH units or more preferably within ±0.5 dKH units.

The dKH of the growth medium may be varied by varying the amount of carbonate and/or bicarbonate in the growth medium. For example, carbonate and/or bicarbonate may be added to the growth medium to increase the dKH of the medium. In some examples, the growth medium may be dosed with a solution of carbonate and/or bicarbonate. For example, the carbonate and/or bicarbonate may be provided as an alkali metal salt, for instance, a sodium salt. In some examples, the growth medium may be dosed with a solution of sodium carbonate at intervals to maintain the dKH of the growth medium at 8 or more. Dosing may be performed as a bulk powder or as a stock solution.

In some examples, dKH may be monitored. dKH may be measured and/or monitored by any suitable method. For example, the dKH may be measured by manual visual titration, a manual colorimetric method or using an automatic pH electrode. In some examples, the dKH may be monitored continuously or frequently during the period of coral growth.

In addition to controlling the dKH, the concentration of certain elements in the growth medium may also be controlled. For example, the growth medium may be dosed with solutions of one or more salts, for example, metal salts. Examples of suitable salts include salts of magnesium, calcium and strontium. One or more of these salts may be employed. In some instances, metal cations present in the salts may be incorporated into the structure of the growing coral and/or otherwise promote coral growth. For example, the coral may incorporate metalions, for instance, metal ions selected from magnesium, calcium, strontium and mixtures thereof. Suitable salts include chlorides, for example, magnesium chloride, calcium chloride and strontium chloride. Other suitable salts include phosphates, for example, sodium phosphate. In some instances, anions present in the salts may be incorporated into the structure of the growing coral, or otherwise promote coral growth. As an example, phosphate ions present in phosphate salts (e.g. sodium phosphate) may facilitate growth of the coral skeleton.

The growth medium may also be dosed with, for example, iodine.

In some examples, it may be possible to dose iron salts in the growth medium. It has been found, however, that, in some circumstances, iron can increase the rate of nitrification.

The amount of salt(s) and other additives added to the growth medium may be controlled within narrow limits. Accordingly, solutions of the salts/additives may be dosed at predetermined amounts into the growth medium.

In some examples, the concentration of calcium salts (e.g. calcium chloride) may be controlled in the growth medium in an amount of 200 to 500 mg/l, preferably, 350 to 450 mg/l.

In some examples, the concentration of strontium salt (e.g. strontium chloride) may be controlled in the growth medium in an amount of 0 to 12 mg/l, preferably, 6 to 9 mg/l.

In some examples, the concentration of iodine may be controlled in the growth medium in an amount of 25 to 250 mg/l, preferably, 100 to 200 g/l.

In some examples, the amount of phosphate (e.g. sodium phosphate) may be controlled from 0 to 3 mg/l, for example, 0.001 to 1 mg/l.

In some examples, the amount of iron may be controlled from 0 to 3000 mg/l. Preferably, iron concentrations are limited to below 5 mg/l, preferably, below 1 mg/l.

In some instances, no additional iron is added to the growth medium.

The growth medium may also be dosed with ammonium salts. For example, ammonium chloride may be used. Coral has a symbiotic relationship with a dinoflagellate algae of the genus Symbiodinium. This symbiosis is based on mutual nutrient exploitation, with corals providing shelter and inorganic nutrients to their algal partners, while Symbiodinium supply their coral hosts with photosynthates that can meet at least part of the corals' energy requirements. By maintaining ammonium levels in the growth medium, it is possible to support algal growth, which, in turn, facilitates the growth of the coral. By maintaining ammonium levels, it may also be possible to reduce the negative effects of heat stress on the coral. In some examples, ammonium levels are maintained at a concentration of 0 to 1.5 ppm, preferably about 0.5 ppm. Ammonium levels may be maintained by dosing ammonium salts into the growth medium e.g. periodically or continuously. Ammonium levels may be maintained at about 0.5 ppm for e.g. a day. Dosing may be carried out as a bulk powder or as a stock solution.

Where dosing solutions are added to the growth medium, dosing may be carried out manually e.g. at periodic intervals during the growth of the coral. Alternatively, dosing may be carried out automatically e.g. using a peristaltic pump. Dosing may be carried out using a stock solution.

In some instances, live rock may be placed in the growth medium. Coral may anchor to the rock. The rock may also provide a site for nitrifying bacteria within the mesocosm.

The growth medium may be cycled prior to use to ensure that it is stable with nitrifying bacteria. Cycling may be performed according to methods that are well-known in the art.

During the period of coral growth, the temperature of the growth medium may be controlled. Suitable temperatures range from 9 to 35 degrees C., preferably to 32 degrees C., preferably 19 to 32 degrees C. Preferably, the temperatures are controlled within 20 to 27 degrees C., more preferably at about 25 degrees C.

During a daily (24 h) cycle, the light intensity received by the growth medium may be controlled. Suitable light intensity can range from 0 to 300 μmol m⁻² s⁻¹, preferably 20 to 240 μmol m⁻² s⁻¹, preferably 50-120 μmol m⁻² s⁻¹, preferably 60 to 100 μmol m⁻² s⁻¹, preferably 80 μmol m⁻² s⁻¹. The growth medium may also be subjected to a controlled amount of light on a pre-determined cycle as required. The growth medium may be irradiated with light for 4 to 20 hours a day, for example, 6 to 15 hours a day, preferably 8 to 12 hours a day.

The hydrostatic head pressure applied in the growth medium may be controlled during a daily (24 hr) cycle by controlling the depth of coral culture in the culture tanks. Pressure affects the density and pore volume of the coral material. Suitable culture depths range from 0.05 to 1.5 m above the top of the coral in culture, preferably 0.1 to 0.8 m.

The pH of the growth medium may be controlled during the period of coral growth. Suitable pH ranges can be 7 to 9, preferably 7.8 to 8.2, preferably 8.

As detailed above, several factors (such as temperature, light intensity, pressure, pH, carbonate hardness, and the rate of circulation or agitation of the growth medium) may be controlled independently or in combination with one another during the period of coral growth (for example, during a daily (24 h) cycle) to vary the characteristics of the coral. For example, one or more of such growth conditions may be controlled at the suitable ranges provided herein to obtain desired intra-particle pore volumes.

It may take at least one month, for example, two to six months before the coral is ready for harvesting/collection. In some instances, it may take three to four months before the coral is ready for harvesting. In some examples, the coral may be considered to be ready for harvesting once it reaches at least 150%, preferably, at least 180% or at least 200% of its original volume.

Once ready for harvest, the coral may be collected using known methods.

### Devitalising Coral

The collected coral may be devitalised using any suitable method. For example, the coral may be devitalised by treatment with an oxidizing agent, for instance, hypochlorite (e.g. sodium hypochlorite). In one example, the coral may be treated with a 5% solution of hypochlorite for a predetermined length of time. Suitable time periods range from 3 to 50 hours, for example, 5 to 40 hours. If required, the devitalised coral may then be rinsed with water (e.g. deionized water). The collected coral may be dried. Drying may be performed at elevated temperatures. Suitable temperatures range from 50 to 190 degrees C., for instance, 80 to 100 degrees C. In some instances, the collected coral may be subjected to depyrogenation.

The devitalised coral may then be sized using any suitable technique. For example, the devitalised coral may be ground into particles. Where the devitalised coral is sized to form particles of a coral scaffold, sizing may be carried out by crushing or milling. Any suitable crushing or milling technique may be used. For instance, the devitalised coral may be milled e.g. using a disc mill. Fractions can be segregated using wire mesh sieves of various aperture sizing that can conform to ISO 3310/ASTM E11

### Other Treatments

Once sized, the devitalised coral may be used to form the coral scaffold of the bone graft substitute. In some examples, the sized coral may be used as the coral scaffold after, for example, cleaning and disinfection.

In other examples, the coral may be treated prior to use as the coral scaffold. For example, the coral may be treated to convert at least part of the calcium carbonate in the coral to hydroxyapatite. Any suitable method for converting the calcium carbonate in the coral to hydroxyapatite may be employed. For example, the calcium carbonate may be treated by hydrothermal treatment to produce hydroxyapatite. In some examples, the coral may be treated with phosphoric acid (H₃PO₄) a dihydrogen phosphate salt (e.g. ammonium dihydrogenphosphate, NH₄H₂PO₄) to produce hydroxyapatite. The reaction may occur in the presence of water and at elevated temperatures. Suitable temperatures range from, for example, 160 to 220 degrees C. Suitable pressures range from 6 to 10 MPa, for example, 8 MPa.

While the conversion of calcium carbonate to hydroxyapatite may be useful for certain applications, for other applications, it may be preferable for the calcium carbonate to remain unconverted. In some examples, coral scaffolds comprising calcium carbonate may be preferable for use in the manufacture of resorbable bone grafts. In some examples, the coral scaffold is used without treating the scaffold to convert the calcium carbonate to hydroxyapatite.

### Calcium and Calcium Carbonate Content

The coral particles of the bone graft substitute may have a calcium content of greater than at least 35 weight %, preferably at least 37 weight %. In some examples, the coral particles may contain 35 to 45 weight % calcium, preferably 37 to 40 weight % calcium.

Preferably, the coral particles comprises calcium carbonate. The coral particles may comprise at least 85 weight %, preferably at least 90 weight %, more preferably at least 95 weight % calcium carbonate. In some examples, the coral particles comprise at least 98 weight %, preferably at least 99 weight % calcium carbonate. The coral particles may consist essentially of calcium carbonate.

In some examples, at least 95 weight % of the calcium carbonate is present in the aragonite crystalline phase. In some examples, at least 99 weight % of the calcium carbonate is present in the aragonite crystalline phase. For instance, at least 99.5 weight % or at least 99.7 weight % of the calcium carbonate may be present in the aragonite crystalline phase. In some examples, a small amount of calcite may be present. For example, calcite may form up to 1 weight %, preferably up to 0.5 weight %, more preferably up to 0.3 weight % of the calcium carbonate present in the coral scaffold. In some instances, the calcium carbonate consists essentially of calcium carbonate present in the aragonite and/or calcite phase. Crystallinity of the calcium phase may be important, as higher levels of crystallinity may improve the degradability of the bone graft substitute in vivo. Phase quantification may be carried out using any suitable method, for example, by X-ray diffraction as outlined in, for instance, ISO 13779-3:2018.

In some examples, the coral particles may comprise at least 85 weight %, preferably at least 90 weight %, more preferably at least 95 weight % calcium carbonate and at least 95 weight % of the calcium carbonate is present in the aragonite crystalline phase. Preferably, the coral particles comprises at least 98 weight %, preferably at least 99 weight % calcium carbonate and at least 99 weight % of the calcium carbonate is present in the aragonite crystalline phase. For instance, at least 99.5 weight % or at least 99.7 weight % of the calcium carbonate may be present in the aragonite crystalline phase.

In addition to calcium, other metals may be present in the coral particles. For example, the coral particles may comprise sodium, magnesium and/or strontium. Where magnesium is present, magnesium may be present in an amount of up to 2000 mg/kg, for instance, 1000 to 1500 mg/kg, preferably 1100 to 1300 mg/kg. Where sodium is present, sodium may be present in an amount of up to 8000 mg/kg, for example, 4000 to 7000 mg/kg, preferably 5000 to 6000 mg/kg. Where strontium is present, strontium may be present in an amount of up to 9000 mg/kg, for example, 65000 to 8500 mg/kg or 7000 to 8000 mg/kg.

The coral particles may be resorbable in vivo. The coral particles may be resorbable over sufficient time to facilitate bone growth. For example, the particles may be resorbed over at least 6 months, preferably at least 8 months, more preferably at least 10 months in vivo. The particles may be resorbed over at least 12 months, for example, at least 15 months or at least 18 months in vivo. In some examples, the coral particles are resorbed slowly over time to allow sufficient bone remodeling to occur before resorption of the scaffold. This reduces the risk of failure during the healing process, for example, from fracture and/or disunion. This may allow the bone graft substitutes to be used, for example, in load-bearing applications (e.g. spine or long-bone applications).

### EXAMPLES

### Example 1—Particles of Pocillopora

In this Example, the particle size distribution, intraparticle pore volume and overall pore volume of particles of Pocillopora were determined on a fraction size of milled 1-2 mm material. The particles were suitable for use as a coral scaffold for bone growth.

### Particle Size Distribution

To determine the particle size distribution, the sample was sieved for 10 minutes at 1 mm amplitude in a stack of 2.0, 1.4, and 1.0 mm test sieves (from top to bottom) according to a procedure compliant with DIN 66165-2:2016-08. All sieves were qualified according to ISO 3310-1:2016 and ASTM E11. The content of each sieve and the collecting pan was determined by weighing, and relative weight fractions in % of the total weight were calculated.

The particle size distribution of the particles was as shown in the table below.

| Size range (mm) | Weight fraction in % |
|---|---|
| d < 1.0 | 3.6 ± 0.5 |
| 1.0 ≤ d < 1.4 | 32.3 ± 3.4 |
| 1.4 ≤ d < 2.0 | 60.5 ± 3.1 |
| d ≥ 2.0 | 3.6 ± 0.1 |

### Overall Pore Volume

Tapped density was measured according to ISO 23145-1:2007 and using SOP 734255. An aliquot of at least 2 cm³ of sample were filled into a container and tapped 1000 times on a pneumatic device (Bosch Pneumatik BT5). Particles protruding beyond a defined upper edge of the container were scraped off and the weight of the granules inside the container was determined on an analytical scale. Finally, the tapped density was obtained by division of the weight by the volume of the test container (1.4 cm³).

The porosity, P, i.e. the percentage of void space in the tapped granule aliquot, was calculated from the tapped density, ρ_{tapped}, as follows:

$$P = ( 1 - \frac{{ρ}_{t a p p e d}}{{ρ}_{t h e o r}} ) \times 100$$

where ρ_{theoretica} is the theoretical density of aragonite (2.93 cm³).

Finally, the total volume of sample contained in each product vial was obtained by division of its total weight by its tapped density.

The tapped density of the sample was determined to be 1060±19 g/cm³. The overall pore volume was determined to be 63.8±0.7%.

### Intraparticle Pore Volume

The intraparticle pore volume was determined by mercury intrusion porosimetry (ASTM UOP 578-11). Measurements were taken over a pressure range of 21.33 to 61,000.00 psia and a mercury contact angle of 17.29 degrees.

The sample was found to have an intra-particle pore volume of 1.9996%. The average pore diameter (mean) was 0.17944 μm. The total pore area was 0.203 m²/g.

### Example 2

### SEM Analysis of Pocillopora Coral

The surface topology of the Pocillopora coral was assessed by energy dispersive X-ray (EDX) analysis. The analysis was conducted on a Hitachi S-4700 SEM with EDX Analysis system (Hitachi, Krefeld, Germany). Using INCA software (ETAS Ltd, Derby, UK), a flat area of interest was identified on the external surface of each sample at ×150 magnification.

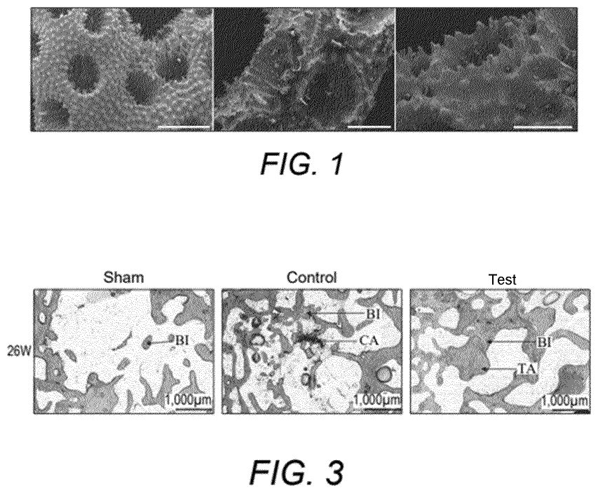

The image is shown in FIG. 1. The left image is taken at ×35 magnification; the middle image at ×60 magnification and the right hand image at ×80 magnification. The Pocillopora scaffold was also characterized by a coarse, textured coenosteum and the presence of tuberculae with uniformly sized calice macro-pores, however they were irregularly spaced along the scaffold.

The highly textured nanotopography of the scaffold may be of clinical importance because surface roughness has a positive correlation with cellular attachment and osteogenic differentiation.

### Example 3

Biocompatibility and Cell Viability with Mesenchymal Stem Cells (MSCs) of Pocillopora Coral

The biocompatibility and cell viability of MSCs cultured adjacent Pocillopora coral scaffolds was investigated.

MSCs from 3 donors were seeded in technical duplicate at a density of 5,263 cells/cm² onto a 96-well plate in MSC expansion medium and incubated at 37° C. and 5% CO₂. After allowing cells to adhere for 24 h, coral was placed in the wells to cover 10% of the well surface area as per ISO 10993-5:2009. Positive control wells contained MSCs cultured in a monolayer without coral. The negative control samples were MSCs without coral, but with the induction of necrosis by the addition of 2% TritonX-100, (Sigma-Aldrich, Wicklow, Ireland). At data collection, the coral scaffolds were removed from each well and the viability of the cells within the well assayed with the LIVE/DEAD Cytotoxicity Kit for mammalian cells (Thermofisher, via Bioscience, Dublin, Ireland) according to the manufacturer's instructions. To visualize the cells, an inverted fluorescent microscope (Olympus IX71) was used with CellSens software using GFP and TRITC filters at an exposure of 580 ms, ISO 1600 and a resolution of 1360×1024.

Fluorescent staining of the positive control, MSCs grown in a monolayer without coral, revealed green-stained cells that retained their characteristic fibroblastic morphology at high viability. Fluorescent staining of the negative control (MSC undergoing induced necrosis) revealed an abundance of rounded, red-stained cells. Imaging the MSCs cultured on the Pocillopora scaffolds revealed green-stained, viable, fibroblastic cells.

### Example 4

### Cell Proliferation of Pocillopora Coral

To determine the proliferative capacity of MSC's when cultured adjacent to devitalised Pocillopora coral, the culture's DNA content was quantified using a Quant-IT PicoGreen dsDNA Assay Kit (ThermoFisher, via Bioscience, Dun Laoghaire) according to the manufacturer's instructions. MSC from 3 donors in technical triplicate were seeded onto a 96-well plate at a density of 5,263 cells/cm² and incubated at 37° C. and 5% CO2 in a humidified incubator. Day 0 samples were harvested 3 hours after plating. After allowing the cells to adhere for 24 h, the coral scaffold was placed in the wells to cover 10% of the well surface. Positive controls consisted of cells seeded onto the plates without the coral scaffolds and negative controls were cells killed by induction of necrosis with 4% TritonX-100 for 20 minutes prior to harvest. DNA content was quantified daily over 4 days of culture with the Quant-iT kit using a Thermo Scientific Varioskan Flash Spectral Scanning Multimode Reader and Skanlt Software 2.4.3 RE for Varioskan Flash. To determine the absolute number of cells in each well, the DNA content was divided by 6 pg {Creane, 2017 #77}. The cell number was normalized to day 0 values. The results are shown below.

| | Test Sample | Positive Control | Negative Control |
|---|---|---|---|
| Day 0 | 1.0 +/− 0.7 | 1 +/− 0 | 0.7 +/− 0.4 |
| Day 1 | 1.6 +/− 0.4 | 2.4 +/− 1.0 | 0.7 +/− 0.4 |
| Day 2 | 2.2 +/− 0.7 | 4.0 +/− 1.9 | 1.1 +/− 0.7 |
| Day 3 | 2.3 +/− 1.1 | 4.2 +/− 2.6 | 0.9 +/− 0.6 |

All data was displayed as a fold increase in cell number normalized to the positive control day 0 and is displayed as an average+/−standard deviation.

The positive control MSCs and MSCs exposed to the coral scaffolds showed a consistent fold increase in cell number over time. In this investigation, MSCs co-cultured with Pocillopora scaffolds remained viable, and exhibited a fibroblastic shape and continued proliferating.

### Example 5

### SEM and Confocal Microscopy of MCSs Grown on Pocillopora

The adhesion, morphology and organization of MSCs grown on the Pocillopora coral were characterized by SEM (FIG. 3) and confocal microscopy.

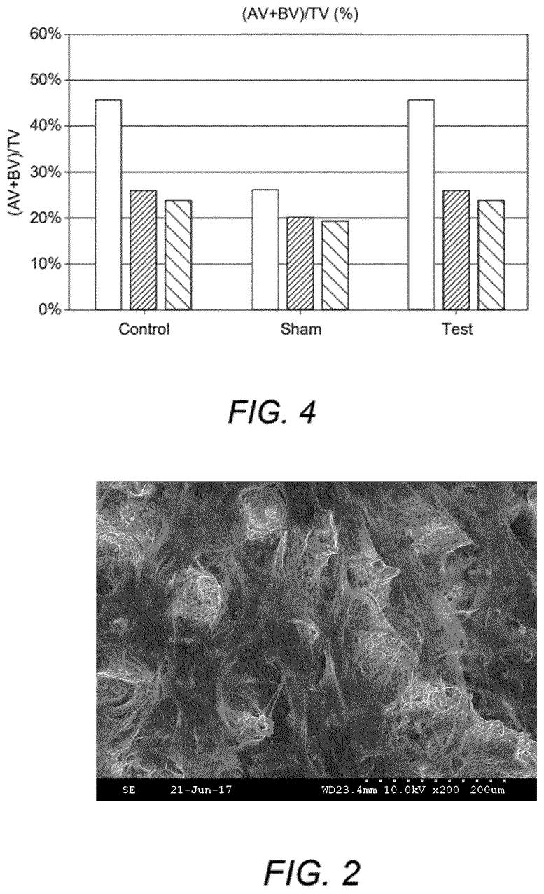

The MSCs on the scaffolds appeared as a dense layer of fibroblastic cells covering the majority of the coenosteum while avoiding the tuberculae. Pseudopods were observed extending from the MSCs, especially across the calice pores where the cells appeared to be cooperating to create a membrane suspended over the concavity (FIG. 2) while still lining the pore. Confocal microscopy of the MSC nuclei revealed the density of cells growing over the coenosteum and into the calice pores while cytoskeletal staining revealed the MSC's elongated, organized, aligned morphology. Large gaps in staining were observed in the confocal imagery where cells avoiding adhering to the tuberculaer structures.

By comparing SEM images of both coral scaffolds with and without the addition of MSCs (FIGS. 1 and 2) it was observed that MSC thickly adhered to the granular surface of the scaffold, covering the majority of the coenosteum, demonstrating the hospitality and biocompatibility of the scaffold. The cells avoided binding to the protruding tuberculae structures on both species of coral. While growing into the concave calices, the MSCs coordinated to physically support one another, creating a suspended membrane across the top of the external calice macropore while at the same time lining the pore. The cells were fibroblastic in shape with extending pseudopods, the latter being indicative of migratory capacity.

### Example 6

### Mechanical Strength

In this example, the compressive strength of coral material of the genus Pocillopora was determined. Coral was grown in captivity at a dKH of 8 and samples were taken from the tip of a coral branch (A) (N=10) and a portion of a branch adjacent the coral core (A′) (N=7). The samples were sized as cylinders having an L/D of 1.5. Their compressive strengths were determined under ISO 9917. A Zwick® Test Tool was used using 5 kN load cell. The loading rate was 1 mm/min.

Sample A was determined to have a mean compressive strength of 23.7 MPa. Samples A′ were determined to have a mean compressive strength of 30.5 MPa.

### Example 7

The purpose of this study was to evaluate the local tissue effects, the degradation and the performance of a void filler consisting of the particles of Example 1 (referred as Test article) in comparison to a commercial synthetic bone void filler based on beta-tricalcium phosphate (Vitoss® Micromorsels, referred to as Control article) after intra-osseous implantation in rabbits.

### Study Design

Forty-nine rabbits were implanted in defects (5 mm of diameter and approximately 10 mm depth) made in the medial femoral condyle of each posterior leg (2 defects per animal). For each rabbit, the defects were filled with the test or control article or were left empty (sham group).

One rabbit was terminated on the day of implantation and its two sites, referred as T0 sites, served to get a baseline for further histopathologic characterisation of the articles and defect initial filing.

Other rabbits were terminated after 4, 13 and 26 weeks of implantation to follow the degradation kinetic of the articles.

Local tissue effects and degradation were evaluated through macroscopic observations and qualitative and semi-quantitative histopathologic analysis. The performance in terms of bone healing and the article degradation was evaluated by histomorphometric and micro-computed tomography analyses.

### Histopathology

At 4, 13 and 26 weeks, histologically, the Test article elicited a null to minimal tissue reaction when compared to the Control article, according to the ISO 10993-6. Both Test and Control articles promoted overall a marked and homogenous bone healing, contrary to the Sham-operated group showing a lower heterogeneous and incomplete bone formation. The Test article showed slower signs of degradation than the Control article at both time periods. Of note, higher signs of bone maturation were qualitatively observed in the Test group at 13 and 26 weeks.

FIG. 3 show representative photomicrographs of the implant/void sites after 26 weeks. Both test (right hand image) and control articles (centre image) promoted respectively a marked and moderate to marked homogenous bone healing, contrary to the sham-operated group (left hand image) showing a lower and heterogenous bone formation (moderate). The bone trabeculae appeared thinner in the control group compared to the test group. The control article was markedly osteointegrated and degraded, while the test article, albeit more extensively osteointegrated, showed lower signs of material degradation (slight). This reduced rate of resorption can reduce the risk of fracture that can sometimes occur when implants are resorbed before osteointegration is complete.

Key: BI (bone ingrowth), CA (control article), DM (defect margin), TA (test article). Histomorphometric Evaluation

Histomorphometrically, after 4 weeks only, the bone-to-implant contact (BIC) value was significantly higher in the Test group vs Control group reflecting a faster bone spreading at the surface of the Test article (osseointegration) compared to the Control article. At 4 and 13 weeks, the bone+article area (%) value was significantly higher in the Test group compared to the sham-operated group only and significantly higher in the Test group vs Control group at 26 weeks. Between 4 and 26 weeks, the rate of material degradation was noticeably higher for the Control group vs Test group.

FIG. 4 compares the bone+article volume % value [(bone volume (BV)+article volume (AV))/total volume (TV)] % for the control article (left hand graphs), sham (middle graphs) and test article (right hand graphs).

The 3D qualitative Micro CT analysis showed at 4 and 26 weeks, that the bone reconstruction was distributed more homogeneously in the Test and Control groups than in the Sham-operated group. The quantitative Micro CT analysis showed at 4 and 13 weeks that the percentage of article+bone volume values (AV+BV/TV) were significantly higher in the Test and Control groups compared to the Sham group. Noteworthy, at 13 and 26 weeks, this value was significantly higher in the Test compared to the Control group and even to the Sham-operated group at 26 weeks.

The Test article did not induce any adverse effects at 4, 13 and 26 weeks following bone implantation.

The osseointegration results (BIC) at 4 weeks and the microscopic (bone maturation) and Micro CT analysis (AV+BV/TV) at 13 and 26 weeks, demonstrated higher signs of bone healing in favour of the Test compared to the two other groups. Much slower degradation was noted with the Test article over time.

1. A bone graft substitute comprising coral particles, wherein coral material of the coral particles has a pore volume of below 15%.

2. A bone graft substitute comprising coral particles, wherein the coral particles comprise coral material of the genus, Pocillopora.

3. A bone graft substitute as claimed in claim 2, wherein the coral material is of the species Pocillopora acuta, Pocillopora aliciae, Pocillopora ankeli, Pocillopora bairdi, Pocillopora brevicornis, Pocillopora capitata, Pocillopora damicornis, Pocillopora danae, Pocillopora effusus, Pocillopora elegans, Pocillopora eydouxi, Pocillopora fungiformis, Pocillopora indiania, Pocillopora kelleheri, Pocillopora ligulate, Pocillopora meandrina, Pocillopora molokensis, Pocillopora verrucose, Pocillopora woodjonesi, and Pocillopora zelli.

4. A bone graft substitute as claimed in claim 1, wherein the coral material of the coral particles has an intra-particle pore volume of below 10%.

5. A bone graft substitute as claimed in claim 4, wherein the coral material of the coral particles has a pore volume of below 5%.

6. A bone graft substitute as claimed in claim 1, wherein the bone graft substitute is in the form of a putty.

7. A bone graft substitute as claimed in claim 1, wherein the bone graft substitute is in the form of a 3D printing composition.

8. A bone graft substitute as claimed in claim 1, which is the form of void filler.

9. A bone graft substitute as claimed in claim 1, which is the form of granules.

10. A bone graft substitute as claimed in claim 9, wherein the bone graft substitute has an overall pore volume comprising an inter-particle pore volume defined by voids between coral particles and an intra-particle pore volume defined by voids within coral particles, said overall pore volume being at least 40%.

11. A bone graft substitute as claimed in claim 10, wherein the overall pore volume is at least 50%.

12. A bone graft substitute as claimed in claim 9, wherein the coral particles have a mean particle size of 1 to 2 mm.

13. A bone graft substitute as claimed in claim 12, wherein greater than 50% of the coral particles have a particle size of 1.4 to 2.0 mm.

14. A bone graft substitute as claimed in claim 1, which further comprises an anti-microbial agent.

15. A bone graft substitute as claimed in claim 1, wherein the coral material has a compressive strength of at least 10 MPa.

16. A bone graft substitute as claimed in claim 1, wherein the coral material has a compressive strength of at least 20 MPa.

17. A bone graft substitute as claimed in claim 1, wherein the coral material has a compressive strength of at least 30 MPa.

18. A bone graft substitute comprising coral particles, wherein the bone graft substitute has an overall pore volume comprising an inter-particle pore volume defined by voids between coral particles and an intra-particle pore volume defined by voids within coral particles, said overall pore volume being at least 40%, and said intra-particle pore volume being below 15%.

19. A bone graft substitute comprising coral particles, wherein the bone graft substitute has an overall pore volume comprising an inter-particle pore volume defined by voids between coral particles and an intra-particle pore volume defined by voids within coral particles, wherein the overall pore volume is at least 3 times greater than the intra-particle pore volume.

20. A method of manufacturing a bone graft substitute according to claim 1, wherein said method comprises:
(i) growing coral in a growth medium;
(ii) removing at least a portion of the coral from the growth medium;
(iii) devitalising coral removed from the growth medium; and
(iv) sizing the devitalised coral to form particles for use as the bone graft substitute.
